(19) United States
(12) Patent Application Publication
(10) Pub. No.: US 2023/0036171 A1
(43) Pub. Date: Feb. 2, 2023

(54) SYSTEMS AND METHODS FOR RAPID VETTING OF COUNSELORS VIA GRADED SIMULATED EXCHANGES

(71) Applicant: Peer Collective Inc., Santa Cruz, CA (US)

(72) Inventor: Timothy Ambrose Desmond, Santa Cruz, CA (US)

(21) Appl. No.: 17/815,735

(22) Filed: Jul. 28, 2022

Related U.S. Application Data
(60) Provisional application No. 63/203,750, filed on Jul. 29, 2021.

(51) Int. Cl. G16H 10/20

(52) U.S. Cl. CPC G16H 10/20

(57) ABSTRACT

Methods and systems for assessing counselors and matching counselors to recipients are disclosed. In some embodiments, a method can include providing, to an applicant, an applicant interface, receiving, from the applicant via the applicant interface, intake data, generating a profile for the applicant, receiving, from the applicant via the applicant interface, at least one simulated exchange response, grading the at least one simulated exchange response to generate a relational intelligence assessment score, generating an assessment of the applicant based at least in part on the at least one score and the intake data, and storing the assessment with the generated profile for the applicant.

### CROSS REFERENCE TO RELATED APPLICATIONS

This application claims the benefit of priority under 35 U.S.C. § 119(e) U.S. Provisional Patent Application No. 63/203,750, filed on Jul. 29, 2021, which is hereby incorporated by reference in its entirety for all purposes.

### BACKGROUND

### Field

This application relates systems and methods for the provisioning of counselors for individual and group counseling.

### Description

Counseling can be useful in a wide variety of settings and can help both individuals and groups. Counseling can be performed in one-on-one or group interactions. Counselors can, among other things, mediate problems between peers, assist with recovery planning and goal setting, assist individuals with navigating support systems and resources, and facilitate support groups. However, conventional methods of matching counselors to recipients can produce poor results as a counselor may not be a good match for a recipient.

### SUMMARY

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

In some aspects, the techniques described herein relate to a computer-implemented method including: providing, to an applicant, an applicant interface; receiving, from the applicant via the applicant interface, intake data; generating a profile for the applicant; receiving, from the applicant via the applicant interface, at least one simulated exchange response; grading the at least one simulated exchange response to generate a relational intelligence assessment score; generating an assessment of the applicant based at least in part on the relational intelligence assessment score and the intake data; and storing the assessment with the profile for the applicant.

In some aspects, the techniques described herein relate to a computer-implemented method, wherein generating an assessment of the applicant includes determining a relational intelligence score.

In some aspects, the techniques described herein relate to a computer-implemented method, further including: in response to receiving one or more ratings of the applicant from one or more service recipients, updating the relational intelligence score, wherein the relational intelligence score is based in part on one or more of the relational intelligence assessment score, a percentage of recipients who rate a first applicant encounter favorably, and a percentage of recipients who rate the first applicant encounter unfavorably.

In some aspects, the techniques described herein relate to a computer-implemented method, wherein grading the at least one simulated exchange response is performed using a machine learning model, and wherein grading the at least one simulated exchange response includes: analyzing the at least one simulated exchange response; and assigning a grade to the at least one simulated exchange response based on the analyzed at least one exchange response.

In some aspects, the techniques described herein relate to a computer-implemented method, wherein there are at least two simulated exchanges responses, wherein at least one simulated exchange response is graded by a human grader, and wherein generating a relational intelligence assessment score includes analyzing the at least one simulated exchange response that is graded by the human grader.

In some aspects, the techniques described herein relate to a computer-implemented method, further including: recommending, based at least in part on the assessment, the applicant to a first recipient having a first recipient assessment; facilitating a first interaction between the applicant and the first recipient; and receiving information indicative of the first interaction.

In some aspects, the techniques described herein relate to a computer-implemented method, wherein the information indicative of the first interaction includes any combination of one of more of video of the first interaction, audio of the first interaction, a transcript of the first interaction, and feedback from the first recipient.

In some aspects, the techniques described herein relate to a computer-implemented method, wherein the recommending is performed using a counselor recommendation model, wherein the counselor recommendation model includes a machine learning model configured to recommend counselors based any combination of one or more of a first recipient assessment, a first recipient acquisition path, relational intelligence scores, and counselor survey data.

In some aspects, the techniques described herein relate to a computer-implemented method, wherein the counselor recommendation model is further configured to: receive counseling session scores; and retrain the counselor recommendation model using the counseling session scores.

In some aspects, the techniques described herein relate to a computer-implemented method, further including: recommending the applicant to a second recipient having a second recipient assessment, wherein the second recipient assessment indicates that the second recipient has a characteristic that is different from a characteristic of the first recipient; facilitating an interaction between the applicant and the second recipient; receiving information indicative of the second interaction; and updating the counselor recommendation model based at least in part on the information indicate of the second interaction.

In some aspects, the techniques described herein relate to a system including: a non-transitory computer-readable medium with instructions encoded thereon; and one or more processors configured to execute the instructions to cause the system to: provide, to an applicant, an applicant interface; receive, from the applicant via the applicant interface, intake data; generate a profile for the applicant; receive, from the applicant via the applicant interface, at least one simulated exchange response; grade the at least one simulated exchange response to generate a relational intelligence assessment score; generate an assessment of the applicant based at least in part on the relational intelligence assessment score and the intake data; and store the assessment with the profile for the applicant.

In some aspects, the techniques described herein relate to a system, wherein generating an assessment of the applicant includes determining a relational intelligence score.

In some aspects, the techniques described herein relate to a system, wherein the instructions, when executed by the one or more processors, further cause the system to: in response to receiving one or more ratings of the applicant from one or more service recipients, update the relational intelligence score, wherein the relational intelligence score is based in part on the relational intelligence assessment score, a percentage of recipients who rate a first applicant encounter favorably, and a percentage of recipients who rate the first applicant encounter unfavorably.

In some aspects, the techniques described herein relate to a system, wherein grading the at least one simulated exchange response is performed using a machine learning model, and wherein to grade the at least one simulated exchange response, the system is configured with instructions to: analyze the at least one simulated exchange response; and assign a grade to the at least one simulated exchange response based on the analyzed at least one simulated exchange response.

In some aspects, the techniques described herein relate to a system, wherein there are at least two simulated exchanges responses, wherein at least one simulated exchange response is graded by a human grader, and wherein generating a relational intelligence assessment score includes analyzing the at least one simulated exchange response that is graded by the human grader.

In some aspects, the techniques described herein relate to a system, wherein the instructions, when executed by the one or more processors, further cause the system to: recommend, based at least in part on the assessment, the applicant to a first recipient having a first recipient assessment; facilitate a first interaction between the applicant and the first recipient; and receive information indicative of the first interaction.

In some aspects, the techniques described herein relate to a system, wherein the information indicative of the first interaction includes any combination of one of more of video of the first interaction, audio of the first interaction, a transcript of the first interaction, and feedback from the first recipient.

In some aspects, the techniques described herein relate to a system, wherein the recommending is performed using a counselor recommendation model, wherein the counselor recommendation model includes a machine learning model configured to recommend counselors based any combination of one or more of a first recipient assessment, a first recipient acquisition path, relational intelligence scores, and counselor survey data.

In some aspects, the techniques described herein relate to a system, wherein the counselor recommendation model is further configured to: receive counseling session scores; and retrain the counselor recommendation model using the counseling session scores.

In some aspects, the techniques described herein relate to a system, wherein the instructions, when executed by the one or more processors, further cause the system to: recommend the applicant to a second recipient having a second recipient assessment, wherein the second recipient assessment indicates that the second recipient has a characteristic that is different from a characteristic of the first recipient; facilitate an interaction between the applicant and the second recipient; receive information indicative of the second interaction; and update the counselor recommendation model based at least in part on the information indicative of the second interaction.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 is a schematic diagram illustrating an example embodiment of a system for rapid applicant assessment of counseling applicants.

FIG. 2 is a schematic diagram illustrating an example embodiment of a system for automated matching of counselors to service recipients.

FIG. 3 is a flowchart illustrating an overview of an example embodiment of a rapid assessment of a counseling applicant using simulated exchanges.

FIG. 4 is a flowchart illustrating an overview of an example embodiment of matching a service recipient to a counselor using a rapid assessment and recommendation system.

FIG. 5 is a flowchart illustrating an overview of an example embodiment of retraining models used to perform automated assessment and matching of counseling applicants to service recipients.

FIG. 6-8 illustrate example features of an example embodiment of a system for rapid applicant assessments and recommendations.

FIG. 9 is a flowchart illustrating an example process for training a machine learning model.

FIG. 10 is a diagram of an example computer system configured for use with some embodiments of the systems and methods described herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of some specific embodiments of the inventions. In addition, embodiments of the inventions can comprise several novel features and no single feature is solely responsible for its desirable attributes or is essential to practicing the inventions herein described.

Counseling can provide valuable benefits to people who are experiencing a wide variety of issues such as anxiety, depression, anger management, grieving the loss of a loved one, coping with trauma or posttraumatic stress disorder (PTSD), or dealing with a crisis. However, there are several barriers to effective counseling. Matching a recipient to an appropriate counselor can be challenging, as it is important that the recipient be matched with a counselor that they feel comfortable talking with, that can understand the recipient's issues, and so forth. If recipients are poorly matched to counselors, the recipient may not derive as much benefit from counseling, become frustrated, abandon counseling, and so forth. Additionally, it is important to perform adequate screening and information gathering prior to a counselor being matched with recipients. Some prospective counselors may not be suited to providing counseling services, for example because they don't show a desired level of empathy or because their approach to counseling differs from the type of counseling contemplated by a company or organization that facilitates counseling services.

Typically, counselors are screen based on, for example, training, certification, licensure, past experience, and/or live interviews. However, this can be a poor indicator of actual performance in counseling settings. A counselor's actual interactions (or simulated interactions) with counseling recipients can be a better indicator of how a counselor will perform with recipients.

Typically, counselors and recipients are matched based on limited information. A counselor may indicate that they specialize in certain types of issues or have a particular approach to counseling (e.g., more or less guidance, gentler or more aggressive), and a recipient may choose a counselor based on this limited information. Alternatively, a counselor and recipient can be automatically matched, or counselors can view recipients and identify recipients they wish to counsel. However, such approaches can fail to adequately determine whether or not a particular counselor is a good match for a recipient. By focusing on credentials, self-reported specialties, and so forth, recipients may be matched with counselors that cannot show sufficient empathy to the recipient or otherwise struggle to relate to the recipient.

Accordingly, it can be advantageous to develop a better understanding of both counselors and recipients so that better matches can be made between them. This disclosure provides systems and methods for improved matching of recipients and counselors. In some cases, better applicant screen and matching can be achieved by ignoring work history, academic credentials, and so forth, and instead measuring a prospective counselor's ability to build helping relationships by grading simulated exchanges. That is, it can be advantageous to determine a prospective counselor's emotional intelligence and ability to respond appropriately to counseling needs.

Simulated exchanges can be designed to assess how the applicant performs in a broad range of situations. For example, simulated exchanges can include skeptical clients, highly personal questions, grief, confrontations (e.g., the client says they feel worse after speaking with the counselor), substance abuse, and so forth.

In some embodiments, counselors, recipients, or both can undergo rigorous screenings and evaluations prior to being matched. In some embodiments, a system can be trained over time using feedback from past counseling sessions, information about different recipients, and so forth, to better match recipients and counselors. For example, a system can be trained to recognize that a first recipient is similar to a second recipient (e.g., similar counseling needs, similar personality, and so forth) and can recommend that the first recipient use a counselor that the second recipient indicated was a good match, or the system can recommend counselors similar to the second counselor. Such an approach can help identify strengths, weaknesses, preferences, and so forth that may otherwise go undiscovered.

Advantageously, such a system can help counselors develop over time. For example, if a counselor appears weak in one area, the counselor could take additional training to develop stronger skills. In some cases, a counselor may engage in practice or mock sessions to develop skills and/or to evaluate their present skills and identify potential areas for improvement.

This disclosure describes systems and methods for providing rapid assessments of counseling applicants and matching of counselors to counseling recipients. In particular, in some embodiments as described herein, systems and methods are provided for assessing counseling applicants (hereinafter referred to as “applicants”) based on graded simulated exchanges. In these embodiments, applicants participate in a plurality of simulated exchanges with counseling subjects, and the applicants' performance during the simulated exchanges is graded or assessed according to a set of metrics. In some embodiments, the metrics may include a relational intelligence score. In some embodiments, the grading of applicants may be performed manually. In these embodiments, the manual grading data may be further processed by a computer system using an algorithm or machine learning model to further develop applicant assessments. In other embodiments, grading may be performed by a machine learning system. In some embodiments, the metrics used to assess applicant performance may be updated over time to incorporate data based on, for example, the success of prior applicants and prior applicant assessments.

In some embodiments, the systems described herein may match counselors to service recipients. In these embodiments, the service recipients may be individuals, companies, or other groups seeking counseling services. In some embodiments, the system may generate a profile of the service recipient. The recipient profile may be based on service recipient data including responses to intake assessment questions given to the service recipient. For example, a recipient may indicate the type of challenge they're facing, how it is affecting them, the style of support they're looking for (e.g., someone to listen, someone to help with direct problem solving, someone to challenge their views or offer new perspectives, and so forth), or characteristics of a desired counselor (e.g., gender identity, age, race, sexual orientation, and so forth)

In some embodiments, the system may perform further processing to develop a recipient profile, including using an algorithm or machine learning model to analyze service recipient data. The system may use the profile to identify particular counselor assessment metrics of lesser or greater relevance to the service recipient. The systems may identify, based on counselor assessments, counselor candidates optimally suited to meet the needs of the service recipient. In some embodiments, an algorithm may be used to identify optimal counselor candidates. Additionally or alternatively, the system may use a machine learning model to identify optimal counselor candidates. In some embodiments, the system may present service recipients with a plurality of profiles identified as optimal matches for their service needs. In some embodiments, the system may receive data in the form of feedback from service recipients to update applicant performance metrics.

Some embodiments described herein are directed to providing rapid assessment of individuals who apply to be counselors (e.g., peer counselors) and matching of service recipients to counselors. In some embodiments, the system can be configured to automatically grade and assess an applicant based on their performance in a number of simulated counseling interactions according to data-driven metrics. For example, a counseling applicant may watch a series of short videos and submit recorded responses, which may then be graded by a machine learning algorithm according to previously determined metrics. Without these metrics, service recipients and counseling providers have to rely on conventional evaluation criteria in assessing an applicant, such as the applicant's level of certification, training, or licensure, the applicant's past experience, or live interviews. These conventional criteria have a number of weaknesses. For example, they are poor predictors of an applicant's future performance in counseling particular service recipients. This may lead to negative outcomes, including poor counseling performance, reputational damage to providers, and discouraging service recipients from seeking counseling in the future.

While some examples herein relate specifically to peer counseling, the systems and methods disclosed herein are not limited to peer counseling. Rather, the systems and methods herein can be used in a wide variety of circumstances where it is advantageous to match a service provider with service recipients based on how well the provider and recipient are likely to match. Indeed, the systems and methods described herein can be applied to a variety of fields where context-specific emotional intelligence is useful, such as management, sales, healthcare, and customer service.

As discussed herein, “counselor,” “peer counselor,” “peer counseling applicant,” or “applicant” is defined to include any peer counselor, therapist, manager, or other person who provides services to individuals or groups in the fields of mental health, psychology, education, or business. Services can be provided in individual settings, group settings, or both. As discussed herein, “service recipient” or “recipient” is defined to include any individual or group who receives services from a counselor as defined above.

Some embodiments of the systems and methods described herein are directed at addressing the shortcomings of conventional assessments. In particular, in some embodiments described herein, providers may take advantage of automated assessment of counselors and targeted matching to prospective service recipients to ensure a better quality of applicants and optimal selection of counselors to meet service recipients' needs.

Applicant Assessment

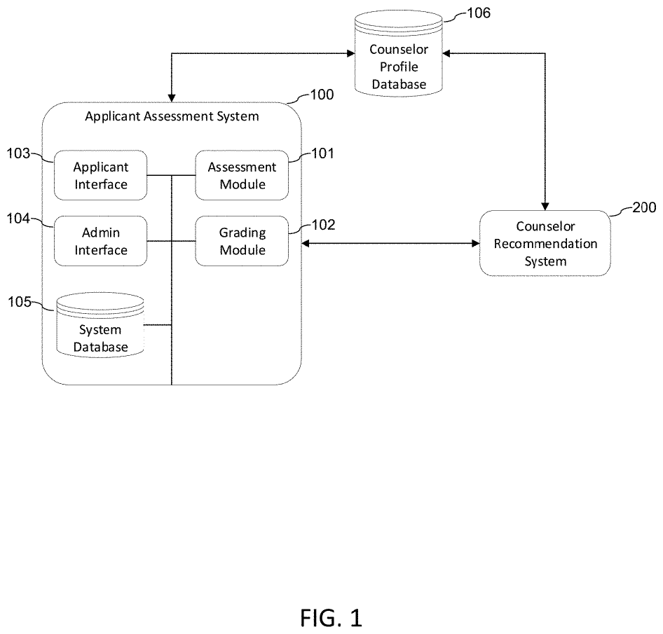

As shown in FIG. 1, in some embodiments a system may comprise an applicant assessment system 100, which can include an assessment module 101, a grading module 102, an applicant interface 103, an administrator interface 104, and a system database 105. In some embodiments, the applicant assessment system can be used to screen applicants and to make hiring decisions. In some embodiments, the applicant assessment system 100 may be connected to a counselor profile database 106 and a counselor recommendation system 200. In some embodiments, the applicant assessment system 100 may comprise software configured to run on a computer system, such as a desktop computer, a server, or a cloud computing environment. In some embodiments, the various components of the applicant assessment system may comprise individual programs or code modules communicatively coupled within a distributed computing environment.

In some embodiments, the applicant interface 103 may comprise an interface for applicants to interact with the applicant assessment system to provide intake data for generating an applicant profile and perform simulated exchanges. In some embodiments, intake data may comprise biographical information, resume or curriculum vitae information, and answers to intake questions. In some embodiments, this information may be stored locally in system database 105 until a counselor profile is created. In some embodiments, one or more transformations may be applied to the intake data, for example to convert the intake data to a standardized format. For example, the applicant's age, date of birth, name, address, and so forth may be transformed to conform to a particular format (e.g., “YYYY-MM-DD,” “MM-DD-YYYY”, etc. for dates, middle names may be stored as a full middle name or as an initial, suffixes and/or prefixes can be added, deleted, or reformatted, and so forth). In some embodiments, an applicant may submit one or more credentials such as a degree or certification, and the system may be configured to communicate with a plurality of third-party services to receive information for verifying the credential information. In some embodiments, the system may convert the verification information to a standardized format. In some embodiments, the system may verify the user's identity by communicating with one or more third-party services. In some embodiments, the applicant interface 103 may be further configured to allow applicants to update and/or view their profile information, search for clients, and/or receive feedback from existing or former clients. In some embodiments, the applicant interface 103 may comprise one or more web applications configured to enable applicant interactions. For example, the applicant interface may comprise a web page from which an applicant may submit, view, and update profile information and intake data. An example web application may be further configured to include a video player and video recorder to enable applicants to perform simulated exchanges from a home computer.

In some embodiments, the administrator interface 104 may comprise an interface for administrators of the applicant assessment system 100 to perform system maintenance, inspect system information, and adjust system parameters. The administrator interface 104 may comprise a web application, a computer program, or a graphical user interface. In some embodiments, administrators may use the administrator interface 104 to update metrics and models associated with the assessment module 101 or the grading module 102, update data associated with simulated exchanges such as videos of the exchanges, update and inspect applicant profile information, and review applicant responses to simulated exchanges. In some embodiments, the administrator interface 104 may be further configured to allow administrators to export applicant profile information, including data stored in system database 105 and/or counselor profile database 106. For example, an administrator may use the administrator interface 104 to export applicant responses to simulated exchanges for manual grading or review.

In some embodiments, system database 105 may be configured to store settings and other data related to the operation of the application assessment system 100. In some embodiments, the system database 105 may further store information related to the assessment module 101 and grading module 102, such as machine learning models, scoring information, etc. In some embodiments, the system database 105 may also be configured to store data related to simulated exchanges, including videos presented to applicants and applicant responses. In some embodiments, the system database 105 may be further configured to store applicant profile information before it is copied to the counselor profile database 106.

In some embodiments, grading module 102 may provide grading of applicant responses to simulated exchanges. In some embodiments, the grading module may provide an interface for administrators or human graders to input grades associated with applicant responses. In other embodiments, the grading module 102 may further comprise a machine learning program or engine trained to automatically grade applicant responses. In these embodiments, the grading module 102 may use the machine learning engine to analyze applicant responses to simulated exchanges and assign grades to applicants based on the output of each analysis. In some embodiments, the machine learning engine may be further configured to analyze additional information, including grades from one or more human graders, in generating grades for applicant responses. In some embodiments, the machine learning engine may use models that are updated based on applicant performance over time. For example, an applicant may be graded by the machine learning engine, and feedback from service recipients who have received services from the counselor may be incorporated into the machine learning model to adjust the grading of future applicants.

In some embodiments, assessment module 101 may be configured to assess an applicant based on at least the applicant's intake data and the applicant's graded responses according to one or more metrics. Metrics can include, for example, whether the applicant communicated articulately and fluently, whether the applicant explicitly put himself or himself in the user's shoes, whether the applicant expressed hope or optimism about building trust with the user, the applicant's emotional expression, whether the applicant seemed authentic (e.g., whether the applicant's words matched their nonverbal expressions), whether the applicant active worked to build alliance with the user, and so forth. The assessment may be based on an algorithm. For example, in some embodiments the assessment module 101 may be configured to calculate a general Relational intelligence (RI-G) score for an applicant. In some embodiments, the assessment module 101 may calculate an RI-G score according to the formula RI-G=(RIA*x)*(GM*y)*(UR*z), wherein RIA represents the applicant's simulated exchange grades (or “Relational Intelligence Assessment” score), GM represents the percentage of past clients who rate the counselor as a “Great Match” after their first encounter, UR represents the percentage of encounters with the counselor that are rated as “Useful,” and x, y, and z are weights adjusted by the system in response to feedback from clients. In some embodiments, x is a constant that does not change over time. In some embodiments, y can increase as the total number of recipients who have completed at least one encounter with counselor increases. In some embodiments, z can increase as the total number of encounters performed by the counselor increases. Thus, for example, after several years of providing services, a counselor's RI-G score can be predominantly derived from the percentage of encounters that are rated as useful by recipients.

In some embodiments, the assessment module 101 may additionally or alternatively use a machine learning engine to determine an applicant's RI-G score. In these embodiments, the machine learning engine may use models that are updated over time based on applicant performance. In some embodiments, the assessment module 101 may be configured to generate a counselor profile including the assessment information. A counselor profile may include the RI-G score of an applicant in addition to other generated assessment information. In some embodiments, for example, the assessment module 101 may generate further assessment information based on the applicant's intake data. For example, the assessment module 101 may use the applicant's answers to intake questions and biographic data of the applicant to identify particular individuals, groups, or subjects to which the applicant may be best suited to provide services. In some embodiments, the assessment information may further include information gathered from third parties, including grades of previous encounters from other recipients of the applicant's services. In some embodiments, the applicant assessment system 100 may subsequently store the counselor profile in counselor profile database 106.

### Counselor Recommendations

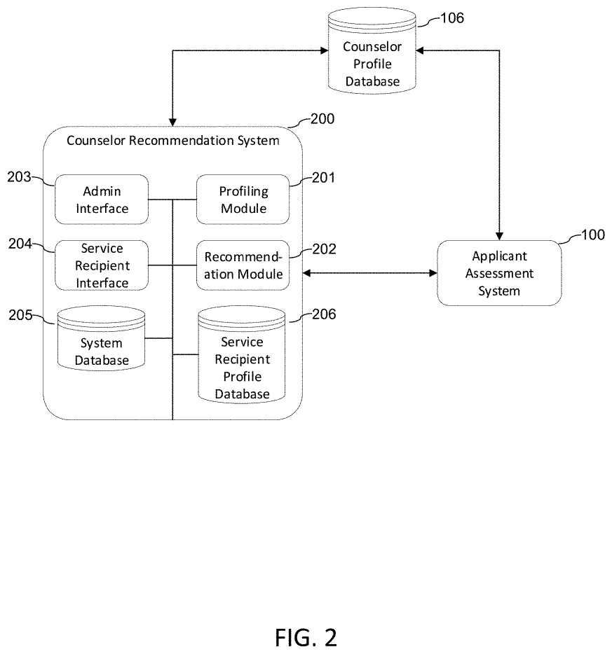

As shown in FIG. 2, in some embodiments the system may comprise a counselor recommendation system 200, which may further comprise a profiling module 201, a recommendation module 202, an administrator interface 203, a service recipient interface 204, a system database 205, and a service recipient profile database 206. In some embodiments, the counselor recommendation system 200 may be connected to a counselor profile database 106 and an applicant assessment system 100. In some embodiments, the counselor recommendation system 200 may comprise software configured to run on a computer system, such as a desktop computer, a server, or a cloud computing environment. In some embodiments, the various components of the counselor recommendation system may comprise individual programs or code modules communicatively coupled within a distributed computing environment.

In some embodiments, the recipient interface 204 may comprise an interface for recipients to interact with the counselor recommendation system 200 to provide intake data for generating a recipient profile and requesting counseling services, select counselors, and provide counselor feedback. In some embodiments, intake data may comprise biographical or organizational information as well as answers to intake questions. Applicant intake data can be manipulated or transformed similarly to the applicant intake data transformations discussed above, for example by converting intake data to standardized date formats, name formats, address formats, and so forth. In some embodiments, this information may be stored in system database 205 until a recipient profile is created. In some embodiments, the recipient interface 204 may be further configured to allow recipients to update and/or view their profile information. In some embodiments, the recipient interface 204 may comprise one or more web applications configured to enable applicant interactions. For example, the recipient interface may comprise a web page from which an applicant may submit, view, and update profile information and intake data. An example web application may be further configured to include a video player and video recorder to enable applicants to perform simulated exchanges from a home computer.

In some embodiments, the administrator interface 203 may comprise an interface for administrators of the counselor recommendation system 200 to perform system maintenance, inspect system information, and adjust system parameters. The administrator interface 203 may comprise a web application, a computer program, or a graphical user interface. In some embodiments, administrators may use the administrator interface 203 to update data and models associated with profiling module 201 and recommendation module 202, update settings and data associated with the counselor recommendation system 200, such as applications and data related to the service recipient interface 204, and review, update, or delete recipient profile information. In some embodiments, the administrator interface 203 may be further configured to allow administrators to export recipient profile information, including data stored in system database 205 and/or service recipient profile database 206. For example, an administrator may use the administrator interface 203 to export recipient profiles for external use.

In some embodiments, system database 205 may be configured to store settings and other data related to the operation of the counselor recommendation system 200. In some embodiments, the system database 205 may further store information related to the profiling module 201 and recommendation module 202, such as machine learning models, algorithms, etc. In some embodiments, the system database 205 may be further configured to store recipient profile information before it is copied to the service recipient profile database 206.

In some embodiments, service recipient profile database 206 may be configured to store profiles of service recipients for subsequent use by the counselor recommendation system 200. In some embodiments, recipient profiles include intake data in the form of biographical or organizational information, answers to intake questions, and profile data generated by profiling module 201. In some embodiments, recipient profiles may further comprise data related to interactions of service recipients with counselors, such as recommendations made by the recommendation module 202, metadata related to counseling sessions between service recipients and counselors, and recipient feedback from counseling sessions including counselor ratings.

In some embodiments, profiling module 201 may be configured to generate recipient profiles based on intake data including biographical or organizational information and answers to intake questions. In some embodiments, the generated profiles may include data related to recipient's counseling needs and preferences. For example, an intake questionnaire may be designed to elicit responses from recipients indicating what kind of counseling they seek, past experiences with counselors, etc. The profiling module may select, based on these responses, one or more codes or metrics relating to the counseling services for which they would be best suited. In some embodiments, answers to questions may be free-form, multiple choice, or indicated by actions taken in response to interactive videos or applications exposed to the recipient via the service recipient interface 204. In some embodiments, the profiling module may further comprise a machine learning engine. In these embodiments, the machine learning engine may be configured to generate profile data based on models incorporating data from past recipients or other sources. For example, the machine learning engine may include a natural language processor configured to analyze responses to free-form intake questions in order to identify recipient metrics. In another example embodiment, the machine learning engine may comprise a neural network trained on profiles of prior recipients and corresponding recipient feedback. In some embodiments, the profiling module 201 may be configured to store generated recipient profiles in the service recipient profile database 206.

In some embodiments, recommendation module 202 may be configured to generate, based on a recipient profile and one or more counselor profiles, a recommendation of one or more counselors for the recipient to select as a service provider. In some embodiments, profiling module 201 may use an algorithm to rank potential providers according to a calculated matching score or relational intelligence (RI) score between each provider and the recipient. For example, an RI score can be calculated as RI=(RI-G)*(RI-S), where RI-G relates to the provider's general relational intelligence, as described above, and RI-S is a specific relational intelligence score that characterizes how well the provider will do in the specific situation presented (e.g., for a specific user).

In some embodiments, RI-S for a specific recipient and provider can be determined by RI-S=a*TGM−b*NGM, where TGM is the body, where TGM is the correlation of all known data points between the specific recipient and all recipients who rated the provider as a “Great Match” after a first encounter, and NGM is the correlation of all known data points between the specific recipient and all recipients who did not rate the provider as a “Great Match” after a first encounter, a is the percentage of users who rated the provider as a “Great Match” after a first encounter, and b=1−a.

Additionally or alternatively, in some embodiments profiling module 201 may use a machine learning engine to rank potential applicants for selection by a recipient. For example, a neural network may be trained on the profile data of applicants and their service recipients, feedback data in the form of counseling outcomes, recipient feedback, and/or feedback of third-party recipients of applicant services. In this example, profile data of an applicant and a recipient may then be analyzed by the machine learning engine to determine a score, based on the past data, indicating the strength of the match.

In some embodiments, machine learning can be used to improve the reliability of RI-S scores for providers with small data sets by using data for other providers. For example, machine learning can be used to determine a similarity rate between a provider with a small data set and other providers. A machine learning model can use, for example, RIA video data (e.g., data from simulated sessions) to calculate similarities between providers. The model can be configured to adjust similarity rates based on correlations of user data for Match/No-Match user segments. For example, if a provider with a small data (Provider Y) set is being compared to Provider Z, similarity rates can be adjusted based at least in part on how similar the Match users are for Provider Y and Provider Z, and/or how similar the No-Match users are for Provider Y and Provider Z. If Provider Z has a large data set of Match and No-Match users, but Provider Y has a small set, the similarity rates can be used to reduce the error in RI-S for Provider Y with respect to a recipient X.

The recommendation module 202 may be further configurable to incorporate likely recipient needs into match calculations. For example, a recipient having complex trauma or negative past experiences may be matched with a top counselor, while recipients who are likely to be easier to treat may not be shown top counselors to choose from. For example, if a recipient is not likely to be difficult to treat, the recommendation module can exclude a portion of counselors based on, RI scores, for example by excluding counselors with RI scores in the top 20%. Conversely, if a recipient's mental health history indicates a need for a top counselor, the recommendation module 202 may, for example, include only applicants with RI scores in the top 20% in its match calculations.

### Example Assessment

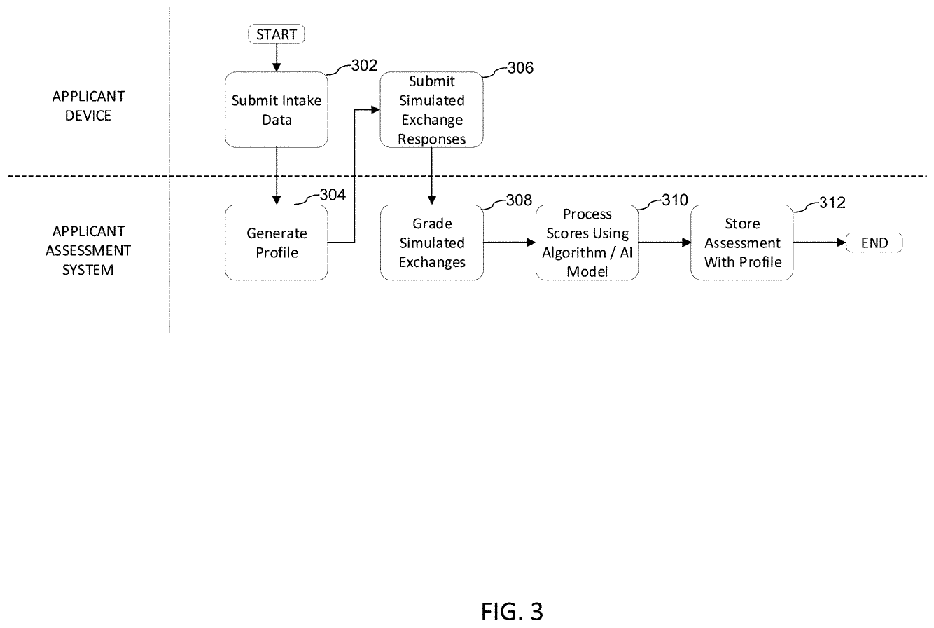

FIG. 3 depicts illustrative interactions between an applicant device and an example embodiment of an applicant assessment system. The interactions begin at 302, in which a new applicant may begin by submitting intake data. In some embodiments, the applicant may use a computing device to submit answers to one or more questionnaires through a web interface or by other means for future upload by an administrator. In other embodiments, the applicant may use a computing device to interact with the applicant assessment system directly via a web application as discussed with respect to FIG. 1 above.

At 304 the applicant assessment system may generate an initial profile of the applicant. In some embodiments, the initial profile may be stored directly with the applicant assessment system, or it may be stored in the counselor profile database.

At 306, the applicant device submits simulated exchange responses. In some embodiments, these may be simulated counseling sessions. In these embodiments, the sessions may be conducted live, in-person, or remotely (e.g., via teleconference, video call, etc.), and may be uploaded to the applicant assessment system by the applicant device. Additionally or alternatively, the applicant may perform the simulated exchanges by using a computing device to interact directly with a web application that plays a series of videos of simulated recipients and records the applicant's responses to each video.

At 308, the applicant assessment system grades the simulated exchanges. In some embodiments, the simulated exchanges may be exported or displayed via an administrator interface for manual grading. In some embodiments, grading may be additionally or alternatively performed by a machine learning engine. In these embodiments, the machine learning engine may be configured to compare the applicant's responses to responses of previously assessed applicants to generate a grade. For example, a first applicant may submit responses bearing a strong similarity to responses of a second, previous, applicant; the machine learning engine may analyze the responses and, judging them similar, assign grades to the first applicant's responses based at least in part on the grades of the second applicant's responses.

At 310, the applicant assessment system generates an assessment of the applicant. In some embodiments, the assessment may be generated based on the applicant's intake data, the applicant's graded simulated exchange responses, and/or third-party data including grades and feedback from previous recipients of the applicant's services. In some embodiments, the assessment may be generated according to an algorithm, as described above with respect to FIG. 1. Additionally or alternatively, the applicant assessment system may analyze the applicant's information using a machine learning engine to generate the assessment data. In some embodiments, the assessment data may include one or more metrics. For example, the assessment data may include a calculation of the applicant's RI score.

At 312, the applicant assessment system stores the assessment data with the applicant profile. In some embodiments, the applicant assessment system will add the completed profile to a counselor profile database. In other embodiments, the applicant assessment system may update an existing profile with the generated assessment data.

### Example Recommendation

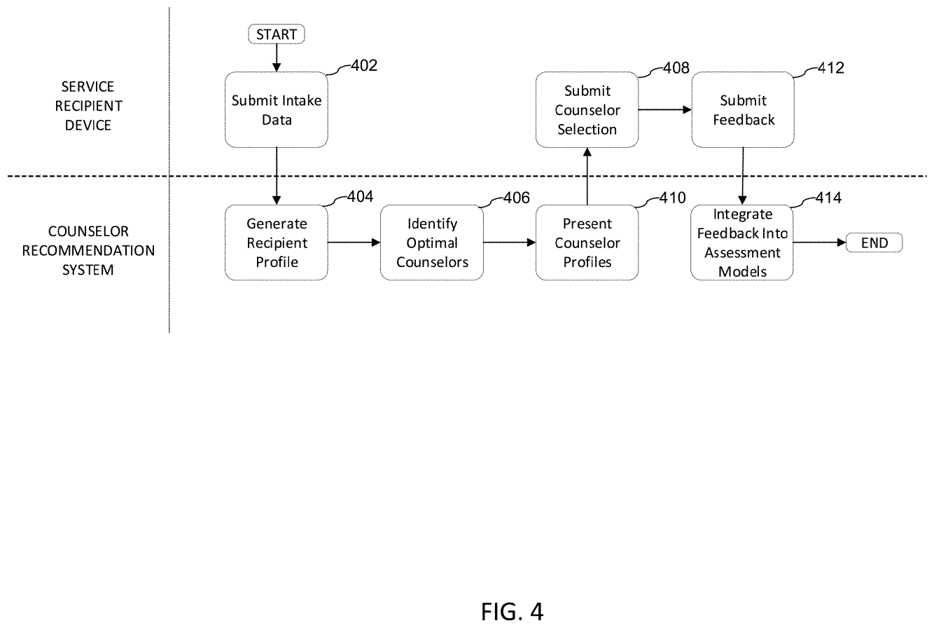

FIG. 4 depicts illustrative interactions between a recipient and an example embodiment of a counselor recommendation system. The interactions begin at 402, in which a service recipient device may begin by submitting intake data. In some embodiments, the recipient may use a computing device to submit a questionnaire through a web interface or by other means for future upload by an administrator. In other embodiments, the recipient device may interact with the counselor recommendation system directly via a web application as discussed with respect to FIG. 2 above.

At 404, the counselor recommendation system may generate a recipient profile. The generated profile may be based on intake data including biographical or organizational information as well as answers to intake questions. In some embodiments, the generated profile may include data related to the recipient's counseling needs and preferences. For example, intake questions may be designed to elicit responses from the recipient indicating what kind of counseling they require, past experiences they have had with counselors, etc. The counselor recommendation system may generate, based on these responses, one or more codes or metrics indicating which counseling services might be most appropriate for the recipient. In some embodiments, the profiling module may further comprise a machine learning engine. In these embodiments, the machine learning engine may be configured to generate profile data based on models incorporating data from past recipients or other sources. For example, the machine learning engine may include a natural language processor configured to analyze free-form responses to intake questions in order to generate recipient metrics. In another example embodiment, the machine learning engine may comprise a neural network trained on profiles of prior recipients and corresponding recipient feedback. In some embodiments, the counselor recommendation system may be configured to store the generated recipient profile in a service recipient profile database.

At 406, the counselor recommendation system identifies one or more counselors optimally suited to provide services to the recipient. In some embodiments, the counselor recommendation system may use an algorithm to rank potential counselors according to a calculated matching score between each counselor and the recipient, as described with respect to FIG. 2 above. Additionally or alternatively, in some embodiments, the counselor recommendation system may use a machine learning engine to generate counselor recommendations. For example, a neural network may be trained on the profile data of applicants and their service recipients, and feedback data in the form of counseling outcomes, recipient feedback, and/or feedback of recipients of applicant services. In this example, profile data of an applicant and a recipient may then be analyzed by the machine learning engine to determine a score, based on the past data, indicating the strength of the match. In some embodiments, the counselor recommendation engine may be further configured to incorporate recipient preferences or needs into match calculations. For example, average users may not need a top counselor, and the counselor recommendation system may exclude those counselors with RI scores in the top 5%, 10%, 15%, 20%, 25%, or any number from about 5% to about 25%, or even more if desired. Conversely, if a recipient's mental health history indicates a need for a top counselor, the counselor recommendation system may, for example, include only applicants with RI scores in the top 20% in its match calculations.

At 410, the counselor recommendation system may present a list of recommended counselors to the recipient. In some embodiments, the list may be transmitted to the recipient, such as via email, fax, mail, or other digital or analog means of communication. In some embodiments, the list may be presented to the recipient via a web interface or web application, as described above with respect to FIG. 2.

Turning now to 408, the recipient device may submit a counselor selection. In some embodiments, the recipient may be restricted to selecting one of a plurality of recommended counselors. In some embodiments, the recipient may be able to additionally or alternatively select from a roster of available counselors ranked according to a plurality of criteria including, for example, the recipient's counseling preferences, counseling subject matter, availability of the counselor, the counselor's past graded encounters, etc.

At 412, the recipient may use a computing device to submit feedback relating to encounters with a counselor, for example whether the counselor was a good match, whether the session was useful, and so forth. For example, the recipient may provide a grade of a counselor indicating “Great Match,” “Not a Great Match,” etc. Additionally, the recipient may provide feedback relating to individual encounters, such as, for example, “Useful” or “Not Useful.” In some embodiments, recipient feedback may further include answers to questionnaires designed to elicit responses regarding other criteria, such as the counselor's demeanor, familiarity with the counseling subject, counseling effectiveness, perceived compatibility with the recipient, etc.

At 414, the recipient feedback is integrated into the matching and/or assessment models. In some embodiments, the feedback may be used to update weights and other variables associated with algorithms used for assessing applicants and matching applicants to recipients. Additionally or alternatively, in some embodiments the recipient feedback may be used to retrain machine learning models to improve the predictive effectiveness of the counselor recommendation system and/or applicant assessment system. In some embodiments, the recipient feedback may be further added to the recipient profile, and feedback specific to encounters or counselors may be added to the respective counselors' profiles.

### Example Retraining of Matching and Assessment Models

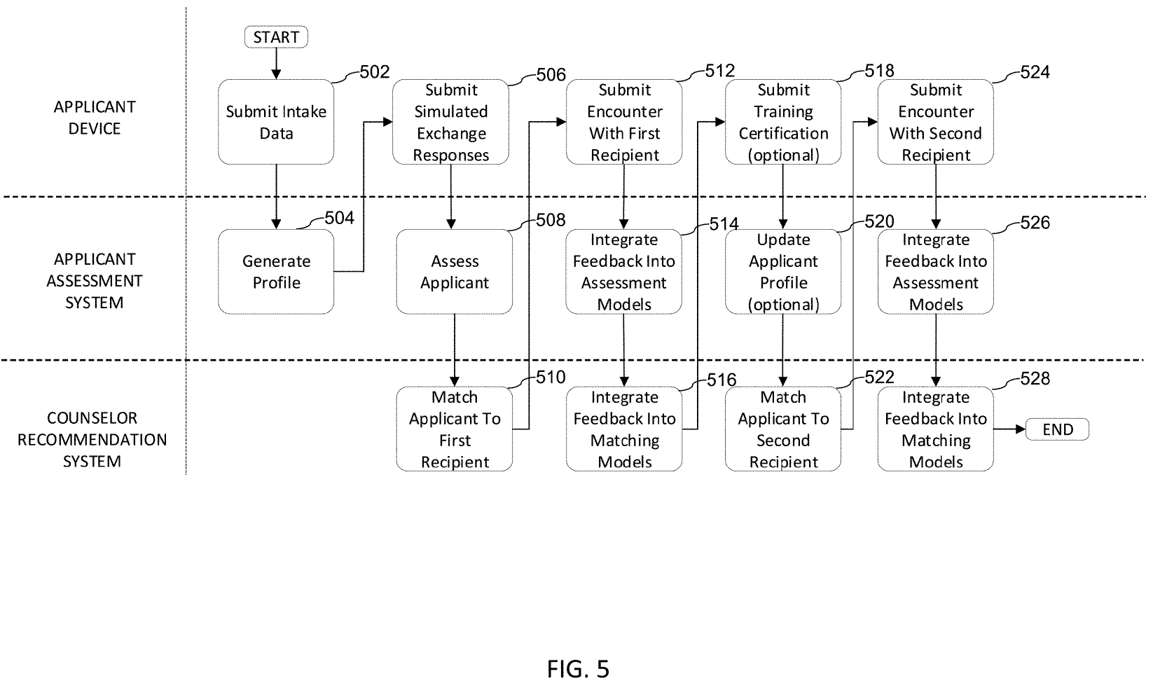

FIG. 5 depicts illustrative interactions between an applicant and an example embodiment of an applicant assessment and counselor recommendation system using machine learning to assess applicants and match them with service recipients. The interactions begin at 502, where an applicant device submits intake data.

At 504, the applicant assessment system generates a profile based on the applicant's intake questions, at 506 the applicant device submits responses to a series of simulated exchanges, and at 508 the applicant assessment system assesses the applicant, as described in more detail with respect to FIG. 3 above.

At 510, the applicant is matched to a first recipient. In some embodiments, this may indicate that a machine learning engine determined that the applicant was recommended as the optimal, or an optimal, counselor to provide services to the first recipient.

At 512, the applicant device submits an encounter with the first recipient. In some embodiments, this may include submitting video and/or audio recordings of a first in-person counseling session with the recipient or a transcript of a session (e.g., transcribed audio, transcribed video, a chat transcript, and so forth). In some embodiments, a transcript can be automatically generated by the system (or an external system) using video and/or audio processing algorithms. In some embodiments, the encounter may be a less formal interaction, such as a video conference, telephone chat, or other initial interaction. In some embodiments, the encounter may come from another system. For example, a counseling platform may record sessions and/or session information in a database, on a file server, and so forth, and may make the encounter data available to the applicant assessment system rather than the encounter data being submitted from the applicant device.

At 514, the applicant assessment system integrates feedback from the first recipient into the assessment models. As described in more detail with respect to FIGS. 1 and 4 above, recipients may provide feedback on counselors and encounters with counselors indicating the quality of the interaction and the counselor's services. In some embodiments, integration of feedback may comprise adjusting the weights associated with an assessment algorithm. For example, an applicant's average feedback score from encounters may be weighted higher as the number of encounters increases for that applicant. In some embodiments, integrating feedback may additionally or alternatively comprise retraining a machine learning model based on the feedback. For example, a counselor with high grades on the simulated exchange performances may have an inconsistently poor encounter with a first recipient. The machine learning models may be retrained, for example, to indicate that counselors similar to the present applicant should have their scores weighted differently or otherwise adjusted to reflect a lower overall assessment. Conversely, a counselor with relatively low grades on the simulated exchange performances may have an inconsistently positive encounter with a first recipient, which may indicate that retraining the model and or re-weighting scores may be warranted. Additionally, in some embodiments, the feedback may be added to the counselor's profile for future use in training models and/or reviewing the counselor's performance.

At 516, the counselor recommendation system integrates feedback from the first recipient into the matching models. In some embodiments, integration of feedback may comprise adjusting the weights associated with a counselor recommendation algorithm. For example, a positive interaction between the applicant and the first recipient may increase the percentage of positive interactions between recipients similar to the first recipient and applicants similar to the applicant, causing the recommendation algorithm to be more likely to recommend a counselor similar to the applicant to future recipients similar to the first recipient.

At 518, the applicant device may optionally submit a training certification. In some embodiments, an applicant's profile may contain recipient feedback indicating deficiencies in one or more areas that an applicant may attempt to rectify by undergoing training. For example, a negative interaction in which an applicant offends a recipient may indicate that an applicant should undergo unconscious bias training in order to avoid similar interactions in the future. If the applicant undergoes training, the applicant assessment system may update the applicant's profile to reflect the training the applicant has undergone at 520.

At 522, the counselor recommendation system matches the applicant to a second recipient. In some embodiments, the second recipient may be significantly dissimilar to the first recipient. In some embodiments, the second recipient may be significantly different at least in part because each recipient chooses their counselor after reviewing the system's suggestions.

At 524, the applicant device submits an encounter with the second recipient. As described with respect to block 512 above, the encounter may be a first counseling session or a less formal interaction.

At 526, the applicant assessment system integrates feedback from the second recipient into the assessment models as described with respect to block 514 above. In the event that the applicant has undergone training between the first and second models, in some embodiments the system may further generate data indicating the effectiveness of the training for applicants of the present applicant's type. In these embodiments, the applicant assessment system may then weight the existence of the training in a future applicant's profile more or less strongly when assessing the future applicant.

At 528, the counselor recommendation system integrates feedback from the second recipient into the matching models as described with respect to block 516 above. In the event that the applicant has undergone training between the first and second models, in some embodiments the system may further generate data indicating that applicants who have undergone the training are more or less appropriate for recipients similar to the second recipient. In these embodiments, the applicant assessment system may then weight the existence of the training in an applicant's profile more or less strongly when assessing the strength of a match with a future recipient.

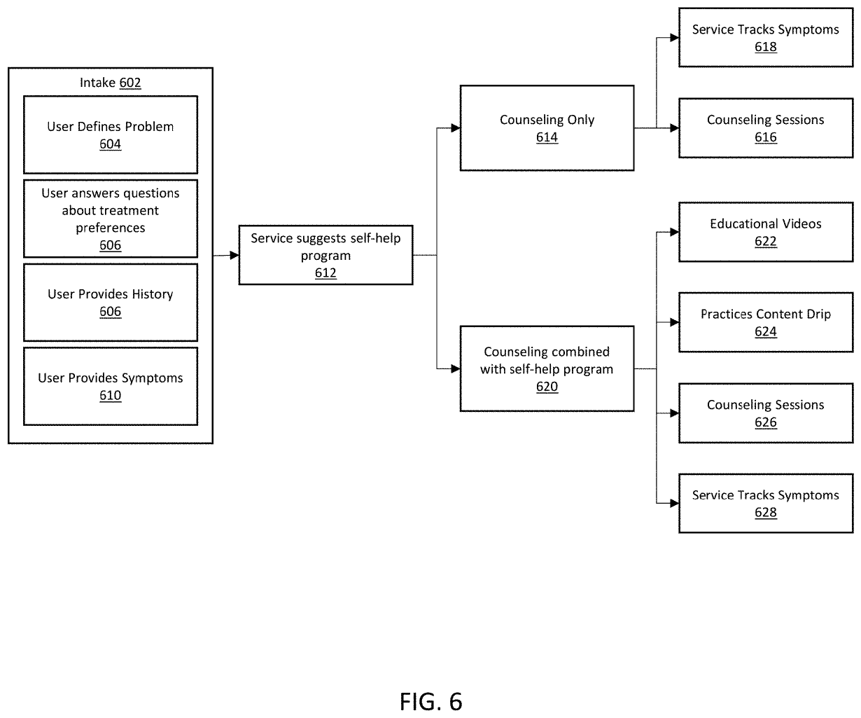

FIG. 6 depicts an example recipient intake and counseling process according to some embodiments herein. FIG. 6 is merely an example process. Other processes may have additional steps, fewer steps, and/or steps may be performed in a different order.

According to FIG. 6, at block 602, a recipient completes an initial intake process. Within the intake process, the user, at block 604, defines the problem or problems for which they are seeking counseling. At block 606, the recipient responds to questions about treatment preferences, such as whether they prefer one-on-one counseling, group counseling, in-person counseling, remote counseling, live counseling, asynchronous counseling, and so forth. At block 606, the recipient can provide historical information and, at block 610, the user can select symptoms they are experienced or have experienced.

At block 612, the system receives the information gathering during the intake process and suggests a self-help program to the recipient. At block 614, the recipient can select to only participate in counseling. The system may facilitate counseling sessions at block 616, and can engage in ongoing symptom tracking at block 618. If instead the recipient chooses to participate in counseling and self-help at block 620, the system can provide educational videos at block 622. At block 624, the system can provide practice content on a periodic (e.g., daily, weekly, or so forth) basis or based on other triggers (e.g., whenever the recipient signs in, before or after viewing an educational video, and so forth). In some embodiments, the practice content can be short (e.g., about 1 minute, 2 minutes, 3 minutes, 4 minutes, 5 minutes, or 10 minutes, or any time from about 1 minute to about 10 minutes) and can be designed to keep the recipient engaged on a regular basis without overly burdening the recipient with content. At block 626, the system facilitates counseling sessions and, at block 628, the system monitors the recipient's symptoms. In some embodiments, a recipient who chooses self-help can share their responses to self-help practices with their counselor. In some embodiments, the system can provide flagged content, viewed content, and so forth to the user and counselor during a counseling session.

In some embodiments, the system may update recommendations, suggest new counselors, and so forth, based on the ongoing monitoring of the recipient's symptoms. For example, if a recipient is not showing progress when engaging only in counseling, the system may suggest that the recipient also participate in self-help programs. In some cases, the system may recommend that the recipient change to a different counselor.

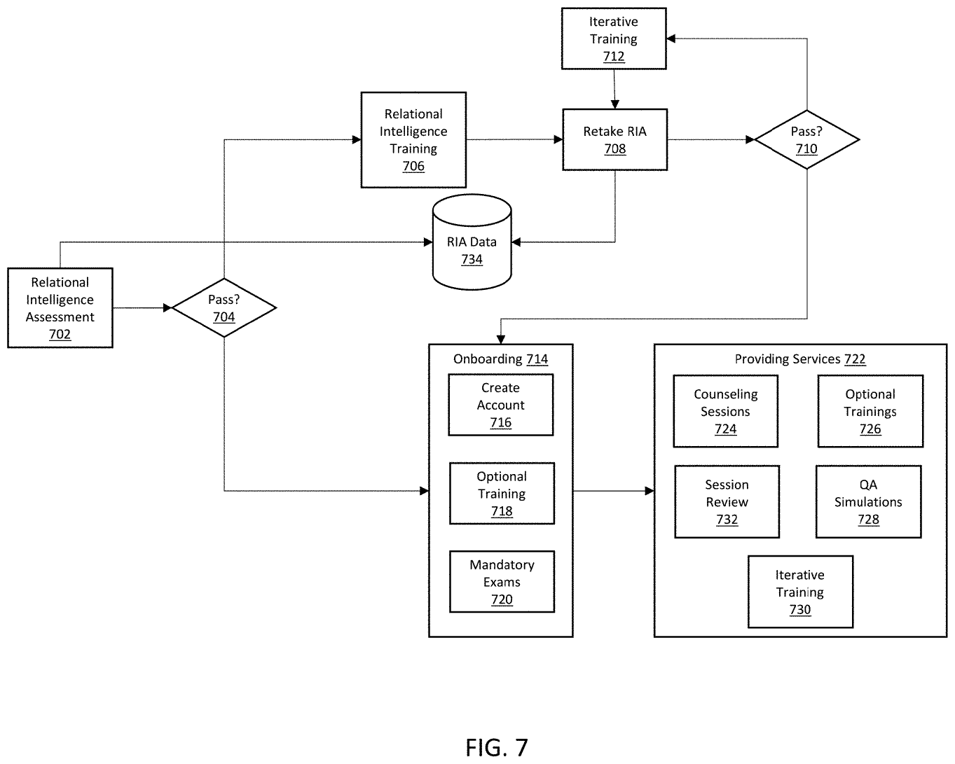

FIG. 7 depicts an example process for screening and monitoring applicants according to some embodiments. In some cases, the process can include more steps, fewer steps, and/or steps may be carried out in an order that is different from that shown in FIG. 7. The process depicted in FIG. 7 may be performed using a computing system.

At block 702, the system can present the applicant with a relational intelligence assessment. At block 704, the system determines whether the applicant met passing criteria for the relational intelligence assessment. If the applicant did not pass the assessment, then the system, at block 706, provides relational intelligence training to the applicant. After completing the relational intelligence training, the applicant retakes the relational intelligence assessment at block 708. At block 710, the system determines whether the applicant passed the relational intelligence assessment. If the user did not pass, then the system may provide the user with additional training at block 712. The applicant, after completing the iterative training at block 712, may retake the assessment at block 708. In some embodiments, the system may allow the user to retake the relational intelligence assessment indefinitely. In other embodiments, however, the system may only permit a limited number of attempts, for example one attempt, two attempts, three attempts, and so forth.

If, at block 704, the user passed the initial relational intelligence assessment at block 702, or if the user passed a retake assessment at block 710, the system can present the applicant with an onboarding process 714. The onboarding process can include several steps, such as account creation 716, optional training 718, and mandatory exams 720. After the onboarding process is complete, the applicant can provide services in block 722.

As shown in FIG. 7, providing services can include several components. The counselor can provide counseling sessions at block 724, participate in optional trainings at block 726, participate in quality assurance simulations at block 728, and receive mandatory training at block 730. At block 732, the system can facilitate review of the sessions conducted at block 724. For example, some sessions may be recorded and an administrator, supervisor, more senior counselor, and so forth can review the counseling sessions. In some cases, the review may be based on surveys submitted by recipients of the counselor's counseling services.

The results of relational intelligence assessments can be stored in data store 734. The results can be used for a variety of purposes, such as monitoring performance, training machine learning models, marketing, and so forth.

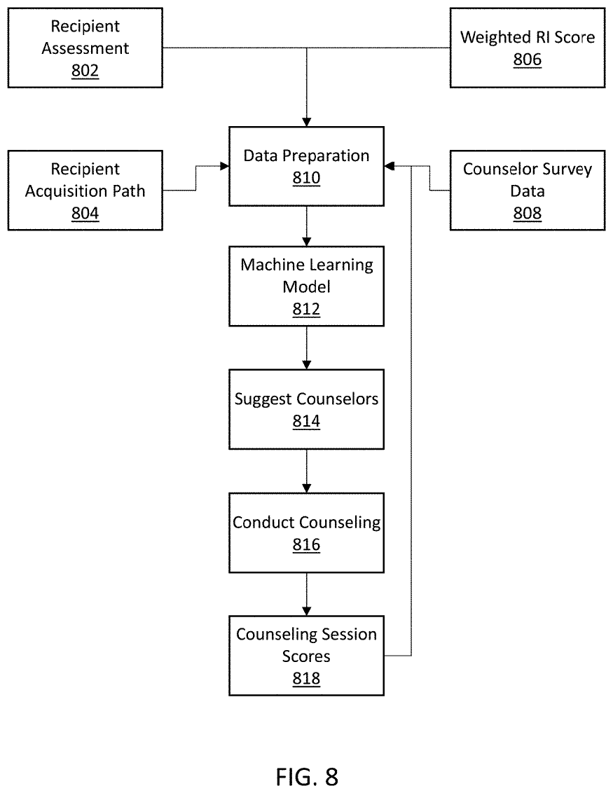

FIG. 8 depicts an example embodiment of a process for training and deploying a machine learning model according to some embodiments herein. In some cases, the process may include more steps, fewer steps, and/or steps may be performed in a different order than presented in FIG. 8. For example, in some embodiments, less, more, or different data may be provided for training a machine learning model. The machine learning model can be deployed on a computer system.

As shown in FIG. 8, recipient assessment data 802 (e.g., demographics, type of issue (substance abuse, trauma, etc.), preferences (training, style, applicant demographics, etc.), and so forth), recipient acquisition path data 804 (e.g., how the recipient navigated to a web site that provides services, who referred the recipient, etc.), RI score data (for the counselor) 806, and counselor survey data 808 are prepared at block 810. At block 812, the machine learning model receives the prepared data and uses the prepared data for, at block 814, suggesting counselors to the recipient. At block 816, the recipient undergoes one or more counseling sessions and, at block 818, provides feedback about the counseling sessions. The feedback at block 818 can be prepared at block 810 and provided to the machine learning model at block 812. The machine learning model can use the feedback to suggest a revised selection of counselors for the recipient to choose from. In some cases, the revised suggestions may be the same as the originally suggested counselors, but in other cases they can be different. For example, if the counseling session scores at block 818 indicate that the recipient found the counseling sessions to be useful and enjoyed working with the counselor, the system may not suggest different counselors. However, if the recipient indicates that the counselor was a bad fit or was ineffective, the system can recommend that the recipient try a different counselor.

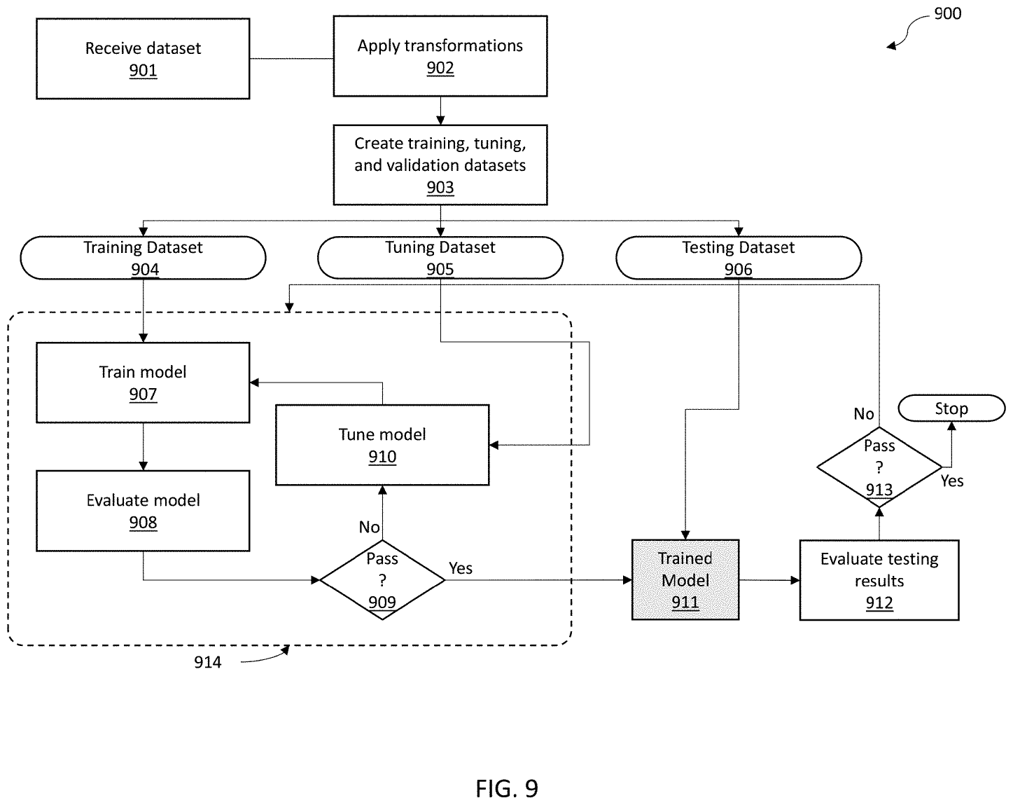

FIG. 9 depicts an example block diagram of a process 900 for training an artificial intelligence or machine learning model according to some embodiments. At block 901, the system may receive a dataset. The dataset may include, for example, recipient assessments, acquisition path data, test scores, counselor survey data, and so forth. At block 902, one or more transformations may be performed on the data. In some cases, dates, times, and so forth may be converted into standardized formats. In some embodiments, categorical data may be encoded in a particular manner and/or nominal data may be encoded using one-hot encoding, binary encoding, feature hashing, or other suitable encoding methods. Ordinal data may be encoded using ordinal encoding, polynomial encoding, Helmert encoding, and so forth. Numerical data may be normalized, for example by scaling data to a maximum of 1 and a minimum of 0 or −1. At block 903, the system may create, from the received dataset, training, tuning, and testing/validation datasets. The training dataset 904 may be used during training to determine variables for forming a predictive model. The tuning dataset 905 may be used to select final models and to prevent or limit overfitting that may occur during training with the training dataset 904, as the trained model should be generally applicable to potential trial participants, rather than tuned to the particularities of patients included in the training dataset 904. The testing dataset 906 may be used after training and tuning to evaluate the model. For example, the testing dataset 906 may be used to check if the model is overfitted to the training dataset. The system, in training loop 914, may train the model at 907 using the training dataset 904. Training may be conducted in a supervised, unsupervised, or partially supervised manner. At 908, the system may evaluate the model according to one or more evaluation criteria. For example, the evaluation may determine how often a recommended counselor is rated as a good fit by users. At 909, the system may determine if the model meets the one or more evaluation criteria. If the model fails evaluation, the system may, at 910, tune the model using the tuning dataset 905, repeating the training 907 and evaluation 908 until the model passes the evaluation at 909. Once the model passes the evaluation at 909, the system may exit the model training loop 914. The testing dataset 906 may be run through the trained model 911 and, at block 912, the system may evaluate the results. If the evaluation fails, at block 913, the system may reenter training loop 914 for additional training and tuning. If the model passes, the system may stop the training process, resulting in a trained model 911.

The training process depicted in FIG. 9 can be deployed in a variety of circumstances. For example, the process (or a similar process) can be used to train a machine learning model to score applicant assessments and can learn over time based on recipient feedback or other inputs, which can enable the model to provide more accurate assessments of applicants. In some embodiments, a model can be trained to recommend counselors to applicants. In some embodiments, a model can be trained the evaluate the effectiveness of self-help program components such as educational videos and/or practice content drips.

### Computer System

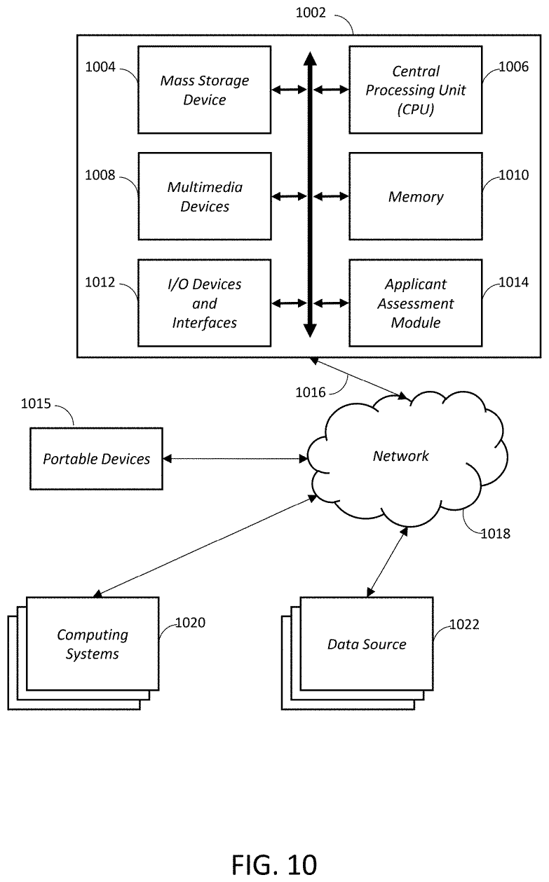

In some embodiments, the systems, processes, and methods described herein may be implemented using one or more computing systems, such as the one illustrated in FIG. 10. The example computer system 1002 is in communication with one or more computing systems 1020 and/or one or more data sources 1022 via one or more networks 1018. While FIG. 10 illustrates an embodiment of a computing system 1002, it is recognized that the functionality provided for in the components and modules of computer system 1002 may be combined into fewer components and modules, or further separated into additional components and modules.

The computer system 1002 can comprise an Applicant Assessment Module 1014 that carries out the functions, methods, acts, and/or processes described herein. The Applicant Assessment Module 1014 is executed on the computer system 1002 by a central processing unit 1006 discussed further below.

In general the word “module,” as used herein, refers to logic embodied in hardware or firmware or to a collection of software instructions, having entry and exit points. Modules are written in a program language, such as JAVA, C or C++, PYTHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems, and may be stored on or within any suitable computer readable medium, or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 1002 includes one or more processing units (CPU) 1006, which may comprise a microprocessor. The computer system 1002 further includes a physical memory 1010, such as random access memory (RAM) for temporary storage of information, a read only memory (ROM) for permanent storage of information, and a mass storage device 1004, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 1002 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1002 includes one or more input/output (I/O) devices and interfaces 1012, such as a keyboard, mouse, touch pad, and printer. The I/O devices and interfaces 1012 can include one or more display devices, such as a monitor, that allows the visual presentation of data to a participant. More particularly, a display device provides for the presentation of GUIs as application software data, and multi-media presentations, for example. The I/O devices and interfaces 1012 can also provide a communications interface to various external devices. The computer system 1002 may comprise one or more multi-media devices 1008, such as speakers, video cards, graphics accelerators, and microphones, for example.

The computer system 1002 may run on a variety of computing devices, such as a server, a Windows server, a Structure Query Language server, a Unix Server, a personal computer, a laptop computer, and so forth. In other embodiments, the computer system 1002 may run on a cluster computer system, a mainframe computer system and/or other computing system suitable for controlling and/or communicating with large databases, performing high volume transaction processing, and generating reports from large databases. The computing system 1002 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 1002 illustrated in FIG. 10 is coupled to a network 1018, such as a LAN, WAN, or the Internet via a communication link 1016 (wired, wireless, or a combination thereof). Network 1018 communicates with various computing devices and/or other electronic devices. Network 1018 is communicating with one or more computing systems 1020 and one or more data sources 1022. Applicant Assessment Module 1014 may access or may be accessed by computing systems 1020 and/or data sources 1022 through a web-enabled user access point. Connections may be a direct physical connection, a virtual connection, and other connection type. The web-enabled user access point may comprise a browser module that uses text, graphics, audio, video, and other media to present data and to allow interaction with data via the network 1018.

Access to the Applicant Assessment Module 1014 of the computer system 1002 by computing systems 1020 and/or by data sources 1022 may be through a web-enabled user access point such as the computing systems' 1020 or data source's 1022 personal computer, cellular phone, smartphone, laptop, tablet computer, e-reader device, audio player, or other device capable of connecting to the network 1018. Such a device may have a browser module that is implemented as a module that uses text, graphics, audio, video, and other media to present data and to allow interaction with data via the network 1018.

The output module may be implemented as a combination of an all-points addressable display such as a cathode ray tube (CRT), a liquid crystal display (LCD), a plasma display, or other types and/or combinations of displays. The output module may be implemented to communicate with input devices 1012 and they also include software with the appropriate interfaces which allow a user to access data through the use of stylized screen elements, such as menus, windows, dialogue boxes, toolbars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 1002 may comprise a physical or logical connection established between a remote microprocessor and a mainframe host computer for the express purpose of uploading, downloading, or viewing interactive data and databases on-line in real time. The remote microprocessor may be operated by an entity operating the computer system 1002, including the client server systems or the main server system, and/or may be operated by one or more of the data sources 1022 and/or one or more of the computing systems 1020. In some embodiments, terminal emulation software may be used on the microprocessor for participating in the micro-mainframe link.

In some embodiments, computing systems 1020 who are internal to an entity operating the computer system 1002 may access the Applicant Assessment Module 1014 internally as an application or process run by the CPU 1006.

The computing system 1002 may include one or more internal and/or external data sources (for example, data sources 1022). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 1002 may also access one or more databases 1022. The databases 1022 may be stored in a database or data repository. The computer system 1002 may access the one or more databases 1022 through a network 1018 or may directly access the database or data repository through I/O devices and interfaces 1012. The data repository storing the one or more databases 1022 may reside within the computer system 1002.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

### OTHER EMBODIMENTS

Although this invention has been disclosed in the context of some embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

Conditional language, such as, among others, “can,” “could,” “might,” or “may,” unless specifically stated otherwise, or otherwise understood within the context as used, is generally intended to convey that some embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present.

What is claimed is:

1. A computer-implemented method comprising:
providing, to an applicant, an applicant interface;
receiving, from the applicant via the applicant interface, intake data;
generating a profile for the applicant;
receiving, from the applicant via the applicant interface, at least one simulated exchange response;
grading the at least one simulated exchange response to generate a relational intelligence assessment score;
generating an assessment of the applicant based at least in part on the relational intelligence assessment score and the intake data; and
storing the assessment with the profile for the applicant.

2. The computer-implemented method of claim 1, wherein generating an assessment of the applicant comprises determining a relational intelligence score.

3. The computer-implemented method of claim 2, further comprising:
in response to receiving one or more ratings of the applicant from one or more service recipients, updating the relational intelligence score, wherein the relational intelligence score is based in part on one or more of the relational intelligence assessment score, a percentage of recipients who rate a first applicant encounter favorably, and a percentage of recipients who rate the first applicant encounter unfavorably.

4. The computer-implemented method of claim 1, wherein grading the at least one simulated exchange response is performed using a machine learning model, and wherein grading the at least one simulated exchange response comprises:
analyzing the at least one simulated exchange response; and
assigning a grade to the at least one simulated exchange response based on the analyzed at least one exchange response.

5. The computer-implemented method of claim 1, wherein there are at least two simulated exchanges responses, wherein at least one simulated exchange response is graded by a human grader, and wherein generating a relational intelligence assessment score comprises analyzing the at least one simulated exchange response that is graded by the human grader.

6. The computer-implemented method of claim 1, further comprising:
recommending, based at least in part on the assessment, the applicant to a first recipient having a first recipient assessment;
facilitating a first interaction between the applicant and the first recipient; and
receiving information indicative of the first interaction.

7. The computer-implemented method of claim 6, wherein the information indicative of the first interaction includes any combination of one of more of video of the first interaction, audio of the first interaction, a transcript of the first interaction, and feedback from the first recipient.

8. The computer-implemented method of claim 6, wherein the recommending is performed using a counselor recommendation model, wherein the counselor recommendation model comprises a machine learning model configured to recommend counselors based any combination of one or more of a first recipient assessment, a first recipient acquisition path, relational intelligence scores, and counselor survey data.

9. The computer-implemented method of claim 8, wherein the counselor recommendation model is further configured to:
receive counseling session scores; and
retrain the counselor recommendation model using the counseling session scores.

10. The computer-implemented method of claim 8, further comprising:
recommending the applicant to a second recipient having a second recipient assessment, wherein the second recipient assessment indicates that the second recipient has a characteristic that is different from a characteristic of the first recipient;
facilitating an interaction between the applicant and the second recipient;
receiving information indicative of the second interaction; and
updating the counselor recommendation model based at least in part on the information indicate of the second interaction.

11. A system comprising:
a non-transitory computer-readable medium with instructions encoded thereon; and
one or more processors configured to execute the instructions to cause the system to:
provide, to an applicant, an applicant interface;
receive, from the applicant via the applicant interface, intake data;
generate a profile for the applicant;
receive, from the applicant via the applicant interface, at least one simulated exchange response;
grade the at least one simulated exchange response to generate a relational intelligence assessment score;
generate an assessment of the applicant based at least in part on the relational intelligence assessment score and the intake data; and
store the assessment with the profile for the applicant.

12. The system of claim 11, wherein generating an assessment of the applicant comprises determining a relational intelligence score.

13. The system of claim 12, wherein the instructions, when executed by the one or more processors, further cause the system to:
in response to receiving one or more ratings of the applicant from one or more service recipients, update the relational intelligence score, wherein the relational intelligence score is based in part on the relational intelligence assessment score, a percentage of recipients who rate a first applicant encounter favorably, and a percentage of recipients who rate the first applicant encounter unfavorably.

14. The system of claim 11, wherein grading the at least one simulated exchange response is performed using a machine learning model, and wherein to grade the at least one simulated exchange response, the system is configured with instructions to:
analyze the at least one simulated exchange response; and
assign a grade to the at least one simulated exchange response based on the analyzed at least one simulated exchange response.

15. The system of claim 11, wherein there are at least two simulated exchanges responses, wherein at least one simulated exchange response is graded by a human grader, and wherein generating a relational intelligence assessment score comprises analyzing the at least one simulated exchange response that is graded by the human grader.

16. The system of claim 11, wherein the instructions, when executed by the one or more processors, further cause the system to:
recommend, based at least in part on the assessment, the applicant to a first recipient having a first recipient assessment;
facilitate a first interaction between the applicant and the first recipient; and
receive information indicative of the first interaction.

17. The system of claim 16, wherein the information indicative of the first interaction includes any combination of one of more of video of the first interaction, audio of the first interaction, a transcript of the first interaction, and feedback from the first recipient.

18. The system of claim 16, wherein the recommending is performed using a counselor recommendation model, wherein the counselor recommendation model comprises a machine learning model configured to recommend counselors based any combination of one or more of a first recipient assessment, a first recipient acquisition path, relational intelligence scores, and counselor survey data.

19. The system of claim 18, wherein the counselor recommendation model is further configured to:
receive counseling session scores; and
retrain the counselor recommendation model using the counseling session scores.

20. The system of claim 18, wherein the instructions, when executed by the one or more processors, further cause the system to:
recommend the applicant to a second recipient having a second recipient assessment, wherein the second recipient assessment indicates that the second recipient has a characteristic that is different from a characteristic of the first recipient;
facilitate an interaction between the applicant and the second recipient;
receive information indicative of the second interaction; and
update the counselor recommendation model based at least in part on the information indicative of the second interaction.
